(19) United States
(12) Patent Application Publication
(10) Pub. No.: US 2011/0023157 A1
(43) Pub. Date: Jan. 27, 2011

(54) EQUINE GENOME EDITING WITH ZINC FINGER NUCLEASES

(75) Inventors: Joseph Bedell, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: Sigma-Aldrich Co., St. Louis, MO (US)

(21) Appl. No.: 12/842,886

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; C12N 5/071; C12N 5/10

(52) U.S. Cl. 800/14; 435/325

(57) ABSTRACT

The present invention provides a genetically modified equine or cell comprising at least one edited chromosomal sequence. In particular, the chromosomal sequence is edited using a zinc finger nuclease-mediated editing process. The disclosure also provides zinc finger nucleases that target specific chromosomal sequences in the equine genome.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified equine or equine cells comprising at least one edited chromosomal sequence. In particular, the invention relates to the use of targeted zinc finger nucleases to edit chromosomal sequences in the equine.

### BACKGROUND OF THE INVENTION

The equine industry is a vital economic component of our economy, which produces horses for aid in work, show, entertainment, racing, rodeo and companionship. Many phenotypic traits associated with horses and breeding have been identified. The genetics of these phenotypes are well documented, but in many cases the actual genes that are responsible are yet to be characterized. The identification of genes controlling several traits of interest in horses has been accomplished by positional candidate cloning. Once the location of a trait is determined by linkage to the markers, possible candidate genes controlling the trait can be inferred because of their proximity to linked markers. Subsets of genes that are mapped in humans and mice have also been mapped in horses through comparative genomic study.

A wide range of assays including DNA typing, infectious disease screening, genetic disorders screening, coat color identification and genetic sex identification are available. Panels for Equine DNA typing are typically based on the internationally accepted panel of microsatallite markers set up by the International Society for Animal Genetics (ISAG). Equine genetic map was published and updated by the Horse Genome Project, available at http://www.uky.edu/Ag/Horsemap/. Other informational databases on the genetic maps of horses have also been done.

In addition to coat color, traits such as elimination of hereditary disease, disease resistance and increased speed are also important for the equine industry. There is a need, therefore, for improved methods of knocking out genes coding undesirable proteins in horses, as well as means of modifying genes involved in desirable phenotypes for higher economic value.

### SUMMARY OF THE INVENTION

One aspect of the present disclosure encompasses a genetically modified equine comprising at least one edited chromosomal sequence.

A further aspect provides an equine embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

Another aspect provides a genetically modified equine cell comprising at least one edited chromosomal sequence.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with equine- or human-related diseases or equine traits. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with equine- or human-related diseases or equine traits generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with equine- or human-related diseases or equine traits using targeted zinc finger nuclease technology is rapid, precise, and highly efficient.

### (I) Genetically Modified Equine

One aspect of the present disclosure provides a genetically modified equine in which at least one chromosomal sequence encoding a disease- or trait- related protein has been edited. For example, the edited chromosomal sequence may be inactivated such that the sequence is not transcribed and/or a functional disease- or trait-related protein is not produced. Alternatively, the edited chromosomal sequence may be modified such that it codes for an altered disease- or trait-related protein. For example, the chromosomal sequence may be modified such that at least one nucleotide is changed and the expressed disease- or trait-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed disease- or trait-related protein comprises a single amino acid deletion or insertion, provided such a protein is functional. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. The modified protein may have altered substrate specificity, altered enzyme activity, altered kinetic rates, and so forth. Furthermore, the edited chromosomal sequence may comprise an integrated sequence and/or a sequence encoding an orthologous protein associated with a disease or a trait. The genetically modified equine disclosed herein may be heterozygous for the edited chromosomal sequence encoding a protein associated with a disease or a trait. Alternatively, the genetically modified equine may be homozygous for the edited chromosomal sequence encoding a protein associated with a disease or a trait.

In one embodiment, the genetically modified equine may comprise at least one inactivated chromosomal sequence encoding a disease- or trait-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional disease- or trait-related protein is not produced. The inactivated chromosomal sequence comprises no exogenously introduced sequence. Such an equine may be termed a “knockout.” Also included herein are genetically modified equines in which two, three, four, five, six, seven, eight, nine, or ten or more chromosomal sequences encoding proteins associated with a disease or a trait are inactivated.

In another embodiment, the genetically modified equine may comprise at least one edited chromosomal sequence encoding an orthologous protein associated with a disease or trait. The edited chromosomal sequence encoding an orthologous disease- or trait-related protein may be modified such that it codes for an altered protein. For example, the edited chromosomal sequence encoding a disease- or trait-related protein may comprise at least one modification such that an altered version of the protein is produced. In some embodiments, the edited chromosomal sequence comprises at least one modification such that the altered version of the disease-related protein results in the disease in the equine. In other embodiments, the edited chromosomal sequence encoding a disease- or trait-related protein comprises at least one modification such that the altered version of the protein protects against a disease or does not form a trait in the equine. The modification may be a missense mutation in which substitution of one nucleotide for another nucleotide changes the identity of the coded amino acid.

In yet another embodiment, the genetically modified equine may comprise at least one chromosomally integrated sequence. The chromosomally integrated sequence may encode an orthologous disease- or trait-related protein, an endogenous disease- or trait-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding an equine or human disease- or trait-related protein may be integrated into a “safe harbor” locus, such as the Rosa26 locus, HPRT locus, or AAV locus. In one iteration of the disclosure, an animal comprising a chromosomally integrated sequence encoding disease- or trait-related protein may be called a “knock-in,” and it should be understood that in such an iteration of the animal, no selectable marker is generally present. An animal comprising a chromosomally integrated sequence encoding an equine or human disease, or trait-related protein may be called a “knock-in.” The present disclosure also encompasses genetically modified animals in which two, three, four, five, six, seven, eight, nine, or ten or more sequences encoding protein(s) associated with a disease or a trait are integrated into the genome.

In an exemplary embodiment, the genetically modified equine may be a “humanized” equine comprising at least one chromosomally integrated sequence encoding a functional human disease or trait-related protein. The functional human disease or trait-related protein may have no corresponding ortholog in the genetically modified equine. Alternatively, the wild-type equine from which the genetically modified equine is derived may comprise an ortholog corresponding to the functional human disease or trait-related protein. In this case, the orthologous sequence in the “humanized” equine is inactivated such that no functional protein is made and the “humanized” equine comprises at least one chromosomally integrated sequence encoding the human disease or trait-related protein. Those of skill in the art appreciate that “humanized” equines may be generated by crossing a knock out equine with a knock in equine comprising the chromosomally integrated sequence.

The chromosomally integrated sequence encoding a disease or trait-related protein may encode the wild type form of the protein. Alternatively, the chromosomally integrated sequence encoding a disease- or trait-related protein may comprise at least one modification such that an altered version of the protein is produced. In some embodiments, the chromosomally integrated sequence encoding a disease or trait-related protein comprises at least one modification such that the altered version of the protein produced causes a disease or forms a trait. In other embodiments, the chromosomally integrated sequence encoding a disease- or trait-related protein comprises at least one modification such that the altered version of the protein protects against the development of a disease or an undesirable trait.

In yet another embodiment, the genetically modified equine may comprise at least one edited chromosomal sequence encoding a disease or trait-related protein such that the expression pattern of the protein is altered. For example, regulatory regions controlling the expression of the protein, such as a promoter or transcription binding site, may be altered such that the disease or trait-related protein is over-produced, or the tissue-specific or temporal expression of the protein is altered, or a combination thereof. Alternatively, the expression pattern of the disease or trait-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a disease or trait-related protein. The genetically modified equine comprising the lox-flanked chromosomal sequence encoding a disease or trait-related protein may then be crossed with another genetically modified equine expressing Cre recombinase. Progeny comprising the lox-flanked chromosomal sequence and the Cre recombinase are then produced, and the lox-flanked chromosomal sequence encoding a disease or trait-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a disease or trait-related protein.

The main determinant of coat color in mammals is the amount and type of melanin pigment in skin and hair. Melanocytes can produce two types of pigment, eumelanin (black/brown) and phaeomelanin (yellow/red), but usually only one pigment type at a time. Binding of α-melanocyte-stimulating hormone (α-MSH) to MSH receptor (MC1-R or MC1R) on melanocyte cell surfaces initiates production of eumelanin. Absence of the α-MSH signal results in phaeomelanin production. Any number of possible alterations to the normal functioning of this complex system will result in horse hair without pigmentation or a dilution of pigmentation. For instance, white color can be caused by several reasons such as the lack of melanocytes or decreased effectiveness of melanin production. Another example is the dominant dark coat color in which a mutation in MC1-R causes activation of this receptor even in the absence of α-MSH.

The coat color of all horses is built on one of two possible base pigments: red or black. The Extension gene controls the production of this base pigment (red or black). All of the coat colors for horses, from white to black, sorrel to gray, begin with one of these two possible base pigments (red or black). All horses will have the genetics for black or red pigment, regardless of their physical appearance. There are a number of dilutions, patterns, and modifiers which a horse can carry that affect the base pigment of a horse.

Horses that are bay, black, grullo, buckskin, black/blue roan, and so forth, are black pigmented horses that carry at least one copy of the Black Factor (E) allele. The black (E) allele of the Extension gene is dominant and causes a black pigmented base both in the heterozygous (Ee) and homozygous (EE) state. A horse that is heterozygous for Red/Black Factor means that it carries one copy of the black allele (E) and one copy of the red allele (e). A horse that is heterozygous for red/black factor can pass on either red or black pigment to its foals. A homozygous black (EE) horse means that it carries two copies of the black allele (EE). A homozygous black horse will always produce black based foals regardless of its mate.

Horses that are chestnut or sorrel, palomino, red dun, red roan, etc. are red pigmented horses and must carry two copies of the Red Factor (e) allele. The red (e) allele of the Extension gene is recessive and will only cause red pigmentation when the horse carries two copies of this allele; this is referred to as Homozygous red (ee). Therefore, a red based foal results when both parents have passed on a copy of the red (e) allele.

Often breeders of black horses will want to test for Red Factor to determine if the horse is homozygous for black (EE). Homozygous black horses will always show black as a base pigment. Thus, the resulting offspring will always be black based and could never be red such as a chestnut or sorrel. Other reasons to test for Red Factor would be to verify that a horse has a red pigment base. Cremello and Palomino horses are homozygous for Red Factor (ee).

The Agouti gene controls the distribution of black pigment. This pigment can be either uniformly distributed or distributed to “points” of the body (ear rims, lower legs, mane, tail). Agouti has been linked to a deletion of 11 nucleotides in the agouti gene. The 11 nucleotide deletion of this gene is the recessive form of the gene. Only when the agouti gene is homozygous for the deletion (aa) is the black pigment evenly distributed. Heterozygous (Aa) or homozygous for the absence of the 11 nucleotide deletion (AA) results in point distribution of black pigment. Agouti has no effect on homozygous positive red factor (ee) horses for there has to be black pigment present for agouti to have an effect. Binding of agouti protein to MC1R causes a switch from eumelanin to phaeomelanin pigment production producing a banding striped pattern in hair color most commonly observed in rodents. Using comparative mapping information, the Agouti gene in equine can be mapped to a particular locus, and the coding region of the equine agouti gene can be isolated.

Studies of the expression and regulation of hair genes are ongoing. Several consensus sequences have been identified within the promoters of these genes, including sequences that bind to transcription factor complexes. The Champagne gene is a recently discovered dominant gene that has the ability to dilute both black and red pigment. Champagne has visual characteristics that differ from cream, pearl and dun dilutions. Some common characteristics of a champagne horse are: pink skin, dark freckles especially around the eyes and on the muzzle, a shiny coat that is often slightly darker in the winter and eye color that will go through a number of color changes starting blue and evolving to a hazel or amber color. It is possible for a horse to have several dilution genes in which a combination of the dilutive affect will be seen. Champagne has been documented in the Quarter Horse, Tennessee Walker, American Saddlebred, Missouri Fox Trotter, Miniature Horses and several other breeds.

Champagne dilution is caused by a dominant gene meaning a single copy of the gene will cause a visibly champagne horse. Unlike cream dilution, there are no visual differences between a horse with one copy or two copies of Champagne. A homozygous champagne horse will always pass one copy of the champagne gene to its foal. Heterozygous horses have a 50% chance of passing the gene on to its foals. Classic Champagne is a black horse with a champagne gene and generally resembles a chocolate color. Gold Champagne is a Chestnut or Sorrel horse plus champagne. Gold Champagne horses also vary in shade and may be registered as sorrel, red dun or palomino depending upon the shade/hue of color. They appear distinctly different though due to the freckling. A bay horse plus champagne is referred to as Amber Champagne. Amber Champagne horses are often confused with buckskin or dun. One difference is that Amber Champagnes generally have brown or even hazel eyes.

Pearl dilution is a recently identified and rare color gene that is thought to have originated in horses of Spanish descent. The presence of the Pearl gene has been confirmed in breeds of Iberian origin, such as the Lusitano and Purebred Spanish horse, and is theorized to be present in the Spanish Mustang. In the American Quarter and American Paint horses, Pearl dilution is regularly referred to as the ‘Barlink Factor.’ Although the gene is distinct, the effect of Pearl dilution is similar to that of the cream and champagne dilutions.

Pearl dilution is a recessive gene, and therefore will only affect the coat of the carrying horse if either two inherited copies of the pearl dilution gene are present or the Cream Dilution Gene is also present in the Pearl-carrying horse. Horses inheriting two copies of the Pearl Dilution Gene will have a diluted coat and pale skin. For example, red horses carrying two copies of Pearl will have a lightened apricot coat, mane and tail, pale skin, and light brown eye color. The combination of the dominant Cream gene and the Pearl dilution gene will produce a pale coat color similar to that of horses that are homozygous for the Cream gene, such as cremello. The skin is pale and the eyes a blue/green color.

The Cream Dilution Gene is one of several genes that will dilute the coat of a horse. Cream is a semi-dominant gene and has a dosage effect where a heterozygous (nCr), single dilute cream horse will show a lesser effect when compared to a homozygous (CrCr), double dilute cream horse. Horses which carry one copy of the cream gene are identified as Single dilutes; they are heterozygous for the cream dilution gene. In the simplest case, a bay horse with a single copy of cream is known as a buckskin, a single dilute black horse is known as a smoky black and a single dilute chestnut or sorrel horse is known as a palomino. Single dilute horses have a 50% chance on passing the cream gene on to its offspring.

Horses which carry two copies of the cream gene are referred to as double dilutes; they are homozygous for the cream dilution gene. A bay horse with two copies of cream is known as a perlino, a black horse with two copies of cream is known as a smoky cream and a chestnut or sorrel horse that carries two copies of cream is known as a cremello. Double dilute horses will always pass on a copy of the cream gene to its foals. Cream dilution is caused by a gene mutation (in this case a single nucleotide polymorphism—SNP) in exon 2 of the MATP gene and subsequently, a genetic test has been developed that tests for the presence of this mutation.

Silver Dilution is a dominant trait. This means, in order to inherit the trait, a horse requires only one parent to carry and pass on the gene. Somewhat similar to the Agouti gene, the Silver Dilution gene will only alter black pigmented horses (Ee or EE) and has no effect on red pigmented horses (ee). The Agouti gene alters the coat by controlling distribution of the black pigment whereas the Silver Dilution gene does so by diluting areas of black pigment.

The effects of the Silver Dilution gene can vary greatly. Dilution by the Silver gene on a horse with a uniform black base typically involves lightening of the mane and tail and a dilution of the body to a chocolate color, sometimes dappled. A Bay horse carrying the Silver gene will usually have a lightened mane and tail, as well as lightened lower legs. It is important to know that although a red horse will not be diluted by the Silver gene, it can however be a carrier of the gene and thus potentially pass the gene on to its offspring. Silver dilution has been identified in a number of horse breeds including the Quarter horse, the Rocky Mountain horse, the Icelandic horse, Morgans, Shetland ponies and the Miniature horse.

Gray (Grey) is a modifier that, over time, causes depigmentation of the horse hair. Horses born with this modifier are born colored but gradually loose pigmentation and can become mostly white in 6-8 years. The vast majority of white horses are in fact horses that have fully grayed out. The Gray modifier is a fully dominant gene meaning a single copy of the gene will cause a visibly graying effect on the base coat. Horses homozygous for the mutation (GG) showed an increased rate of graying as well as more evenly distributed effects during the final stages of graying than a heterozygous gray horse (Gg). Gray occurs in almost every breed although it is more common among a handfull of breeds. Different breeds of horses that commonly show this phenotype are Andalusian, Arabian, Connemara, Iclandic, Lipizzaner, New Forrest Pony, Shetland pony, Thoroughbred and Welsh.

Gray horses, especially horses that are homozygous for the gene, have an extremely high rate of dermal Melanomas (Melanoma Cancer). Research conducted in Sweden showed that 70-80% of gray horses age 15 and older have melanomas. Primary melanomas are generally benign but later metastasize to internal organs. The genetic mutation that produces graying in horses was located in 2008 by researchers at Uppsala University in Sweden. The gray mutation is caused by a 4.6-kb duplication in intron 6 of STX17.

Typically in the heterozygous state, the Sabino coat pattern usually involves a horse having two or more white feet or legs. The white color will often extend up the legs of the horse to the belly in irregular or fragmented patches. These jagged white patches are commonly referred to as barrel spots or belly spots. The head of a Sabino horse is moderately white. It commonly has a blaze or white patch that expands the length of the face. The white areas of a Sabino horse lack pigment, both in the hair and skin. Many Sabinos are characterized by flecks, patches, and roan areas. Roaning (interspersed white hairs throughout the coat) is generally seen around the midsection of the horse. The roaned areas on a Sabino horse can be minimal and only see at the edges of the white pattern to extensive roaning throughout the entire body of the horse. Cases where a Sabino horse exhibits extensive roaning can sometimes be confused with classic roan. However, Classic Roan does not involve the splashy white markings of a Sabino and the head and legs are usually darker than the body. Splashed white is the least common of the spotting patterns in horses, although it is increasing in frequency as breeders use more and more splashed white horses in their breeding programs. However, it has been documented that many splashed white horses are deaf. Recent evidence illustrates that this splashed white pattern and associated deafness is caused by a dominant gene. No homozygous splashed white horses have ever been documented, leading to the conclusion the gene cannot exist in homozygous form. This further indicates that the the loss of hearing probably occurs early in gestation rather than at term, so this is distinct from the lethal white overo (LWO) genetic defect, discussed below.

In the homozygous state, the white often covers greater than 90% of the horse's body. This is referred to as a maximum white sabino. There are instances where a heterozygous horse appears all white but they usually carry another pattern such as Tobiano or Frame Overo. A horse with both Tobiano and Sabino is referred to as a Tovero. Another distinguishing trait among sabinos is their eye color. They commonly have blue eyes or partially blue and brown eyes.

The Tobiano coat pattern usually involves some white on all four legs and rounded white spots on the body with sharp, clean edges. The head of the horse is usually colored and will not have white caused by the Tobiano gene. The white on the body will generally cross the topline of the horse. The Tobiano coat pattern is governed by a dominant gene, meaning that there only needs to be one copy of the gene (tobiano heterozygous-nT) for the tobiano coat pattern to be present.

Homozygosity of the tobiano gene (TT-two copies of the tobiano gene) may show visual clues (“ink spots” or “paw prints”). When there is no presence of the tobiano gene (homozygous negative-nn), the tobiano coat pattern is not possible. several genetic markers that have been found to be strongly linked to the tobiano coat pattern. In 2002, researchers at the University of Kentucky, led by Dr. Samantha Brooks, located a SNP within the KIT gene that is closely linked to the Tobiano pattern. Since then, additional markers have been found within the KIT gene by researchers here at Animal Genetics that we also use to determine Tobiano zygosity. Most recently, researchers at the University of Kentucky discovered a large chromosomal inversion on chromosome 3 near the KIT gene of Tobiano horses. This inversion, which has only been found in Tobiano horses, is believed to disrupt the normal functioning of the KIT gene and cause the Tobiano pattern.

The above represent exemplary examples of equine chromosomal sequences that may be edited include those genes that code for proteins involved in coat color and coat pattern. Non-limiting examples of suitable coat color genes encoding proteins for coat color and pattern include Extension (Black/Red Factor), Agouti, MC1R, Gray Modifier, Champagne Dilution, Tobiano, Silver Dilution, MATP (Cream Dilution), Pearl Dilution, and Sabino1. Those of skill in the art appreciate that other genes and proteins may be involved in coat color and coat pattern, but the genetic loci have not been determined.

In one embodiment, the genetically modified equine may comprise an edited chromosomal sequence encoding the Extension and/or Agouti genes, wherein the edited chromosomal sequence comprises a mutation such that Extension or Agouti is not produced. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that the Extension or Agouti protein is not produced. Thus, a genetically modified equine comprising an inactivated Extension or Agouti chromosomal sequence may produce an alternative desired coat color and pattern.

In another embodiment, the genetically modified equine may comprise an edited chromosomal sequence encoding the Extension (Black/Red Factor), Agouti, MC1R, Gray Modifier, Champagne Dilution, Tobiano, Silver Dilution, MATP (Cream Dilution), Pearl Dilution, and/or Sabinol genes, wherein the edited chromosomal sequence comprises at least one modification such that an altered version of the protein is produced. The modification may be a missense mutation in which substitution of one nucleotide for another nucleotide changes the identity of the coded amino acid. The coding region may be edited to comprise more than one missense mutation such that more than one amino acid is changed. Additionally, the chromosomal region may be modified to have a three nucleotide deletion or insertion such that the expressed protein comprises a single amino acid deletion or insertion, provided such a protein is functional. Those of skill in the art will appreciate that many different modifications are possible in the coding region of the above-disclosed genes. In one embodiment, the genetically modified equine comprising a modified chromosomal region at the location of the above-disclosed genes may have a coat color variation. In other embodiments, the genetically modified equine comprising a modified chromosomal region for the Gray Modifier may have a substantially reduced likelihood of dermal melanoma.

In still another embodiment, the genetically modified equine may comprise an edited chromosomal sequence comprising Extension (Black/Red Factor), Agouti, MC1R, Gray Modifier, Champagne Dilution, Tobiano, Silver Dilution, MATP (Cream Dilution), Pearl Dilution, and/or Sabinol genes, or combinations thereof. The edited chromosomal sequence may comprise at least one modification such that an altered version of Extension (Black/Red Factor), Agouti, MC1R, Gray Modifier, Champagne Dilution, Tobiano, Silver Dilution, MATP (Cream Dilution), Pearl Dilution, and/or Sabinol are produced.

The chromosomal sequence may be modified to contain at least one nucleotide change such that the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified equine comprising an edited Extension (Black/Red Factor), Agouti, MC1R, Gray Modifier, Champagne Dilution, Tobiano, Silver Dilution, MATP (Cream Dilution), Pearl Dilution, and/or Sabinol chromosomal sequence may have a different coat color and/or coat pattern than an equine in which said chromosomal region(s) is not edited.

In yet another embodiment, the genetically modified equine may comprise an edited chromosomal sequence encoding Gray Modifier, Champagne Dilution, Tobiano genes, or combinations thereof. The edited chromosomal sequence may comprise at least one insertion such that an altered version of the Gray Modifier, Champagne Dilution or Tobiano protein is produced.

It is also important for the horse industry to select against a variety of genetic disorders. Hereditary Equine Regional Dermal Asthenia (HERDA) also known as Hyperelastosis Cutis (HC) is a genetic skin disease predominately found in the American Quarter Horse. The symptom of this disorder is a lack of adhesion within the layers of skin due to a genetic defect in the collagen that holds the skin in place. This defect causes the outer layer of skin to split or separate from the deeper layers sometimes tearing off completely. Areas under saddle seem to be most prone to these lesions often leaving permanent scares, preventing the horse from being ridden.

The HERDA disorder is recessive, which means that a horse must be homozygous positive or have two copies of the defective gene to suffer from the disease. Consequently both the sire and the dam must possess at least one copy of the mutated gene in order for the offspring to be afflicted. Offspring born with one copy of the defective gene and one non-defective copy are considered a carrier and have a 50% chance of passing the defective gene on. Therefore the selection against carriers may be a method for decreasing HERDA in horses.

In a further embodiment, the genetically modified equine may comprise an edited chromosomal sequence encoding HERDA, wherein the chromosomal sequence is inactivated such that certain alleles of HERDA protein are not produced. Furthermore, the genetically modified equine having the inactivated HERDA variants chromosomal sequence described herein may exhibit reduced occurrence and carriers of HERDA. In a non-limiting embodiment, the genetically modified equine may comprise an edited chromosomal sequence encoding HERDA. In another non-limiting embodiment, the genetically modified equine may comprise an edited chromosomal sequence inactivating HERDA only in the forms of variants that are known to be carriers.

Hyperkalemic Periodic Paralysis Disease (HYPP) is a muscular disease that affects both horses and humans. In horses, HYPP has been traced back to one horse named Impressive and has the alternative name, Impressive Syndrome, named after this horse. Symptoms of HYPP may include muscle twitching, unpredictable paralysis attacks which can lead to sudden death, and respiratory noises. Severity of attacks varies from unnoticeable to collapse or sudden death. The cause of death is usually respiratory failure and/or cardiac arrest. The HYPP gene is dominant so both homozygous positive (HH) and heterozygous (nH) will cause this muscular disorder. Only homozygous negative (nn) has no HYPP effect. Since HYPP is dominant, the effects of it can also be transposed to other species of horses when intermixing occurs. This makes the recognition and elimination of this disorder very important in preserving the inherited health of all horses.

In a further embodiment, the genetically modified equine may comprise an edited chromosomal sequence encoding HYPP, wherein the chromosomal sequence is inactivated such that the HYPP dominant allele and protein are not produced. Furthermore, the genetically modified equine having the inactivated HYPP dominant allele and chromosomal sequence described herein may exhibit reduced transmittal and perpetuation of HYPP in horses. In a non-limiting embodiment, the genetically modified equine may comprise an edited chromosomal sequence encoding HYPP.

Lethal White Overo (LWO) syndrome occurs when a horse is homozygous (OO) for the frame overo gene. This genetic disorder causes the intestinal system not to develop properly (involving aganglionosis of the bowel). The foal will die within the first 72 hours after birth when its first meals cannot be digested properly. The lethal white foal will be born almost pure white.

This genetic abnormality is caused by a dinucleotide TC-->AG mutation, which changes isoleucine to lysine of the EDNRB protein. Horses that do not have LWO syndrome can still be carriers of the LWO gene. When they are carriers of this gene, they are heterozygous (nO) for the LWO gene and may pass it on to offspring. The heterozygous LWO gene in a horse occurs when the diploid (one copy from mother and one from father) of the LWO gene contains one frame overo copy and one non-frame overo copy and is often referred to as positive for frame overo. Lethal White Overo (LWO) syndrome occurs when a horse is homozygous (OO) for the frame overo gene. This genetic disorder causes the intestinal system not to develop properly (involving aganglionosis of the bowel). The foal will die within the first 72 hours after birth when its first meals cannot be digested properly. The lethal white foal will be born almost pure white.

Breeders breeding two overo horses (heterozygous nO) can expect a 50% chance of producing an overo foal, a 25% chance of producing a lethal white foal OO and a 25% chance of producing a non-overo foal. Breeders breeding a frame overo horse (heterozygous nO) with a non-overo horse (homozygous negative nn) can expect a 50% chance of producing an overo foal nO and no chance of producing a lethal white foal OO. There is a rare possibility of there being another mutation within this gene that causes lethal white overo. Even when a horse has been tested nn, it still has the rare possibility of carrying this other mutation that causes lethal white overo.

In another embodiment, the genetically modified equine may comprise an edited chromosomal sequence encoding the Overo protein, wherein the chromosomal sequence is inactivated such that certain alleles of the Overo protein are not produced and/or are not fatal, but are still able to produce a frame overo phenotype. In a non-limiting embodiment, the genetically modified equine may comprise an edited chromosomal sequence encoding Overo wherein the dominant allele is inactivated. In another non-limiting embodiment, the genetically modified equine may comprise an edited chromosomal sequence inactivating Overo only in the forms of variants that are known to be carriers. In yet another embodiment, the modification changes the dinucleotide TC-->AG mutation, to revert the mutation back to isoleucine in the EDNRB protein.

Glycogen branching enzyme deficiency (GBED) is a disorder first recognized by clinicians at the University of Minnesota that causes muscle weakness in Quarter Horse and related breeds. The clinical presentation of this disease is variable. Late term abortion or stillbirth is described for GBED. Recent research suggests that at least 3% of abortions in Quarter horses are due to GBED. Some foals are born alive but are often weak and require warming and assistance to nurse after birth. These foals may appear healthy for a time but eventually the may develop seizures, become too weak to stand, or in some cases, they die suddenly. Owners may note that GBED foals are less active than other foals. In spite of aggressive treatment, all known cases of GBED have been euthanized or died by 18 weeks of age.

The discovery of an abnormal sugar within the skeletal muscle of these foals led the researchers to identify a genetic defect (glycogen branching enzyme gene) responsible for forming the sugar (glycogen) that provides energy for numerous tissues in the body. GBED is inherited in horses, just as in human beings. GBED is an autosomal (non-sex cell) recessive disease. This means that horses can be carriers and not show signs of the disease, but have affected offspring. Foals with disease receive an abnormal allele (copy) from both the dam and the sire. In horses with GBED, there is a mutation in the Glycogen Branching Enzyme (GBE) gene on chromosome 26 that terminates protein synthesis.

In a further embodiment, the genetically modified equine may comprise an edited chromosomal sequence encoding GBE, wherein the chromosomal sequence is inactivated such that the GBE recessive allele is inactivated and protein not produced. Furthermore, the genetically modified equine having the inactivated GBE variants may exhibit reduced occurrence and carriers of GBE. In a non-limiting embodiment, the genetically modified equine may comprise an edited chromosomal sequence encoding GBE. In another non-limiting embodiment, the genetically modified equine may comprise an edited chromosomal sequence inactivating GBE only in the forms of variants that are known to be carriers.

JEB is an abbreviation for Junctional Epidermolysis Bullosa which is the new name for the old disease called E.I. (Epitheliogenesis Imperfecto) or hairless foal. This genetic mutation results in the defective production of a skin protein that holds the skin to the body. The foals are typically born alive and well, but soon develop patches of hair and skin loss over points of wear. These patches soon become larger and encompass large areas of the foal's body. Hoof attachment is also dependent on this protein and with its absence leads to the loss of the hoof wall. The foal dies or is euthanized for severe infection and discomfort at 3 to 8 days of age. The foals also have another important feature and that is the front teeth are in at birth with many oral ulcers also present. Lethal JEB foals (jj)-these horses die shortly after birth and never reproduce. Carrier horses (Jj)-these horses carry the genetic mutation and it is the breeding of two carrier horses that allows the birth of a JEB foal. Non-carrier horses (JJ)-these horses do not carry the genetic mutation and the breeding of two non-carrier horses results in all their offspring being non-carriers.

In another embodiment, the genetically modified equine may comprise an edited chromosomal sequence encoding JEB, wherein the chromosomal sequence is inactivated such that the JEB recessive allele is inactivated and protein not produced. Furthermore, the genetically modified equine having the inactivated JEB variants may exhibit reduced occurrence and carriers of JEB. In a non-limiting embodiment, the genetically modified equine may comprise an edited chromosomal sequence encoding JEB. In another non-limiting embodiment, the genetically modified equine may comprise an edited chromosomal sequence inactivating JEB only in the forms of variants that are known to be carriers.

Polysaccharide Storage Myopathy (PSSM) is a disease that occurs when excess glycogen (storage form of glucose) or glucose-6-phosphate (form of glucose taken into cells) is present in muscles. Often referred to as PSSM, this disease is called equine polysaccharide storage myopathy, or EPSM, when it occurs in draft horses. A clinical diagnosis of PSSM can be made when a horse that suffers from chronic exertional rhabdomyolys (ER) is found to have amylase-resistant polysaccharide in his muscles. Amylase is the enzyme that breaks starches down to sugars. ER is a syndrome of muscle pain and cramping that is associated with exercise. The PSSM mutation is inherited in a dominant fashion, meaning that one copy of the mutation can cause PSSM. This is different from diseases such as HERDA and GBED, which are inherited in a recessive fashion, where 2 copies of the mutant gene are required for disease.

In a further embodiment, the genetically modified equine may comprise an edited chromosomal sequence encoding PSSM, wherein the chromosomal sequence is inactivated such that the PSSM dominant allele and protein are not produced. Furthermore, the genetically modified equine having the inactivated PSSM dominant allele and chromosomal sequence described herein may exhibit reduced transmittal and perpetuation of PSSM in horses. In a non-limiting embodiment, the genetically modified equine may comprise an edited chromosomal sequence encoding PSSM.

Additionally, horses with a specific mutation in the myostatin (MSTN) have improved athletic performance. Researchers' data has revealed that horses homozygous for the MSTN-C variant (C/C) were more likely to win short races up to 6.5 furlongs (1.3 kilometers), whereas horses with two copies of the MSTN-T (T/T) variant did better in longer races of up to 13.5 furlongs while the heterozygotic horses (C/T) generally did best at intermediate distances. Predictably, mass-to-height ratio was significantly related to allele type, especially for 2-year-old colts, which are typically larger and more muscular than fillies of the same age: C/C males had a 6.7% greater mass per cm than T/T colts. Further, the team found that C/C and C/T horses matured earlier and typically began their racing careers as 2-year-olds. In yet a further embodiment, the genetically modified equine may comprise anedited chromosomal sequence including the C/C, C/T or T/T variant for speed and/or athletic performance, depending on the nature of the desired phenotypic trait.

The present disclosure also encompasses a genetically modified equine comprising any combination of the above described chromosomal alterations. For example, the genetically modified equine may comprise an inactivated agouti and/or edited PSSM chromosomal sequence, a modified MATP chromosomal sequence, and/or a modified or inactivated JEB chromosomal sequence.

Recurrent exertional rhabdomyolysis (RER) is the most prevalent muscle disease in Thoroughbred horses. It has been estimated that 5-10% of racing Thoroughbreds develop exertional rhabdomyolysis during a racing season with recurrences of up to 17%. This disorder has an autosomal-dominant mode of inheritance with variable expression. RER is believed to be caused by an abnormality in intracellular calcium regulation during muscle contraction. However, important genes involved in the regulation of myoplasmic calcium such as ryanodine receptor 1 (RyR1), sarcoplasmic reticulum calcium ATPase (ATP2A1), and dihydropyridine receptor-voltage sensor (CACNA1S) genes were excluded from linkage to RER. Using linkage and comparative genome analysis, the gene relating to RER generally can be located and isolated.

SCID is a deadly diseased gene affecting horses (primarily Arabians) all over the world. SCID (Severe Combined Immunodeficiency Disorder) is a recessive disease gene that is inherited, passed on to new foals from carrier parents. The defect in the B-lymphocyte system has been shown in these affected horses by hypogammaglobulinemia, lymphopenia, and absence of germinal centers. It is believed the cause is generally related to a mutation in DNA-PKcs (DNA-dependent protein kinase catalytic subunit) which results in defective coding and signal joint formation. Other genes that may have causal relation are still under investigation.

Recurrent uveitis as a sequela to Leptospira infection is the most common infectious cause of blindness and impaired vision of horses worldwide. Leptospiral proteins expressed during prolonged survival in the eyes of horses with lesions of chronic uveitis include LruA and LruB. Uveitic eye fluids contained significantly higher levels of immunoglobulin A (IgA) and IgG specific for each protein than did companion sera, indicating strong local antibody responses. Moreover, LruA- and LruB-specific antisera reacted with equine ocular components, suggesting an immunopathogenic role in leptospiral uveitis. Autoantigens were identified by analyzing the autoantibody binding pattern from horses affected by equine recurrent uveitis. Cellular retinaldehyde-binding protein (cRALBP) is such an autoantigen in equine recurrent uveitis horses, which induces B and T cell autoreactivity and established a link to epitope spreading.

Cerebellar abiotrophy is a cerebellar syndrome characterized by degeneration of Purkinje cells. The lesion is due to an intrinsic abnormality in the metabolic structure of these neurons, which preclude their survival. The clinical signs appear at different times after birth and are rapid or slowly progressive. The history and genetic analysis indicate that the disease is due to an autosomal recessive gene, the identity of which is yet under investigation.

Lavender Foal Syndrome (LFS) is a lethal inherited disease of horses with a suspected autosomal recessive mode of inheritance. LFS has been primarily diagnosed in a subgroup of the Arabian breed, the Egyptian Arabian horse. The condition is characterized by multiple neurological abnormalities and a dilute coat color. Candidate genes that lead to LFS include the ras-associated protein RAB27a (RAB27A) and myosin Va (MYO5A). Exon sequencing of the MYO5A gene from an affected foal revealed a single base deletion in exon 30 that changes the reading frame and introduces a premature stop codon.

Additionally, the human or equine disease- or equine trait-related gene may be modified to include a tag or reporter gene, as is well-known. Reporter genes include those encoding selectable markers such as cloramphenicol acetyltransferase (CAT) and neomycin phosphotransferase (neo), and those encoding a fluorescent protein such as green fuorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalamal), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

The genetically modified equine may be heterozygous for the edited chromosomal sequence or sequences. In other embodiments, the genetically modified equine may be homozygous for the edited chromosomal sequence or sequences.

As used herein, equine shall mean any animal in the genus Equus, including but not limited to various species within the genus Equus, such as Equus caballus, Equus asinus, Equus ferus, Equus quagga, Equus zebra, and Equus grevyi, and/or any crossbred offspring hybrid variations of these animals, whether fertile or infertile. The genetically modified equine may be a member of any equine breed. As used herein, the term “equine” encompasses embryos, fetuses, newborn foals, juveniles, and adult equine organisms. In each of the foregoing iterations of suitable equines for the invention, the equine does not include exogenously introduced, randomly integrated transposon sequences.

### (II) Genetically Modified Equine Cells

A further aspect of the present disclosure provides genetically modified equine cells or cell lines comprising at least one edited chromosomal sequence. The disclosure also encompasses a lysate of said cells or cell lines. The genetically modified equine cell (or cell line) may be derived from any of the genetically modified equines disclosed herein. Alternatively, the chromosomal sequence may be edited in an equine cell as detailed below.

The equine cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The equine cell or cell line may be derived from lung (e.g., AKD cell line), kidney (e.g., CRFK cell line), liver, thyroid, fibroblasts, epithelial cells, myoblasts, lymphoblasts, macrophages, tumor cells, and so forth. Additionally, the equine cell or cell line may be an equine stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Similar to the genetically modified equines, the genetically modified equine cells may be heterozygous or homozygous for the edited chromosomal sequence or sequences.

### (III) Zinc Finger-Mediated Genome Editing

In general, the genetically modified equine or equine cell, as detailed above in sections (I) and (II), respectively, is generated using a zinc finger nuclease-mediated genomic editing process. The process for editing a equine chromosomal sequence comprises: (a) introducing into a equine embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. The embryo used in the above described method typically is a fertilized one-cell stage embryo.

Components of the zinc finger nuclease-mediated method of genome editing are described in more detail below.

### (a) Zinc Finger Nuclease

The method comprises, in part, introducing into an equine embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fokl cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the equine or it may be an exogenous sequence. Additionally, the sequence to be integrated may be operably linked to an appropriate control sequence or sequences. The size of the sequence to be integrated can and will vary. In general, the sequence to be integrated may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 by to about 1000 bp, or more particularly about 700 by to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the equine embryo or cell. Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo or Cell

The method for editing a chromosomal sequence using a zinc finger nuclease-mediated process further comprises culturing the embryo or cell comprising the introduced nucleic acid(s) to allow expression of the zinc finger nuclease.

An embryo may be cultured in vitro (e.g., in cell culture). Typically, the equine embryo is cultured for a short period of time at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the equine species. Routine optimization may be used, in all cases, to determine the best culture conditions for a particular species of embryo. In some cases, a cell line may be derived from an in vitro-cultured embryo (e.g., an embryonic stem cell line).

Preferably, the equine embryo will be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair pathway. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is modified.

The genetically modified equines disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified equines disclosed herein may be crossed with other equines to combine the edited chromosomal sequence with other genetic backgrounds. By way of non-limiting example, suitable genetic backgrounds may include wild-type, natural mutations giving rise to known equine phenotypes, targeted chromosomal integration, non-targeted integrations, etc.

### (IV) Applications

The animals and cells disclosed herein may have several applications. In one embodiment, the genetically modified equine comprising at least one edited chromosomal sequence may exhibit a phenotype desired by humans. For example, inactivation of the chromosomal sequence encoding Agouti may result in equine producing hair with striped color coat. In other embodiments, the equine comprising at least one edited chromosomal sequence may be used as a model to study the genetics of coat color, coat pattern, and/or hair growth. Additionally, an equine comprising at least one disrupted chromosomal sequence may be used as a model to study a disease or condition that affects humans or other animals. Non-limiting examples of suitable diseases or conditions include albinism, hair disorders, and baldness, in addition to Skin diseases such as Hyperelastosis Cutis, or muscular diseases such as Hyperkalemic Periodic Paralysis Disease, Lethal White Overo Syndrome, Glycogen Branching Enzyme Deficiency disorder, and Polysaccharide Storage Myopathy, Recurrent exertional rhabdomyolysis (RER), Severe Combined Immunodeficiency Disorder (SCID). Additionally, the disclosed equine cells and lysates of said cells may be used for similar research purposes.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+FSwiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of Agouti in Model Organism Cells

Zinc finger nuclease (ZFN)-mediated genome editing may be tested in the cells of a model organism such as an equine using a ZFN that binds to the chromosomal sequence of a hair color-related gene of the equine cell such as MSH receptor proteins, agouti signaling protein (ASIP) and melanophilin (MLPH). The particular coat color-related gene to be edited may be a gene having identical DNA binding sites to the DNA binding sites of the corresponding equine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into equine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture may result in mismatches forming between heteroduplexes of the WT and mutant alleles. A DNA “bubble” formed at the site of mismatch may be cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected hair color-related gene locus in equine cells using a ZFN.

### Example 2

### Genome Editing of Agouti in Model Organism Embryos

The embryos of a model organism such as an equine may be harvested using standard procedures and injected with capped, polyadenylated mRNA encoding a ZFN similar to that described in Example 1. The equine embryos may be generally at the one-cell stage when microinjected. Control embryos may be injected with 0.1 mM EDTA. The frequency of ZFN-induced double strand chromosomal breaks may be estimated using the Cel-1 assay as described in Example 1. The cutting efficiency may be estimated using the CEI-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

### Example 3

### Generation of a Humanized Equine Expressing a Mutant Form of Human SCID

The first human mutation in the gene encoding DNA-PKcs (DNA-dependent protein kinase catalytic subunit) has been identified in a radiosensitive T-B-SCID patient. A mutation in the DNA-PKcs gene has been predicted for a long time, but spontaneous mutations had only been identified in mouse, horse and dog models. A single base change at DNA-PKcs may lead to alteration of a disease-associated kinase subunit protein. ZFN-mediated genome editing may be used to generate a humanized equine wherein the equine DNA-PKcs is replaced with a mutant form of the human DNA-PKcs comprising one or more mutations. Such a humanized equine may be used to study the development of the diseases associated with the mutant human DNA-PKcs protein. In addition, the humanized equine may be used to assess the efficacy of potential therapeutic agents targeted at the pathway leading to immunodeficiency comprising DNA-PKcs.

The genetically modified equine may be generated using the methods described in the Examples above. However, to generate the humanized equine, the ZFN mRNA may be co-injected with the human chromosomal sequence encoding the mutant DNA-PKcs protein into the equine embryo. The equine chromosomal sequence may then be replaced by the mutant human sequence by homologous recombination, and a humanized equine expressing a mutant form of the DNA-PKcs protein may be produced.

What is claimed is:

1. A genetically modified equine comprising at least one edited chromosomal sequence encoding an equine or human disease.

2. The genetically modified equine of claim 1, wherein the edited chromosomal sequence is inactivated, is modified, or has an integrated sequence.

3. The genetically modified equine of claim 1, wherein the edited chromosomal sequence comprises a gene chosen from Extension (Black/Red Factor), Agouti, MC1R, Gray Modifier, Champagne Dilution, Tobiano, Silver Dilution, MATP (Cream Dilution), Pearl Dilution, Sabinol, HERDA, HYPP, Overo, GBE, JEB, PSSM, MSTN, RER, DNA-PKcs, cRALBP, MYO5A and combinations thereof.

4. The genetically modified equine of claim 3, wherein the gene is Extension (Black/Red Factor), Agouti, MC1R, or Gray Modifier, or combinations thereof, and the edited chromosomal sequence comprises at least one mutation such that the sequence is inactivated and the associated protein is not produced or is not functional.

5. The genetically modified equine of claim 4, wherein the gene is Gray Modifier and the equine has a reduced likelihood of developing dermal melanoma.

6. The genetically modified equine of claim 3, wherein the gene is Champagne Dilution, Tobiano, Silver Dilution, MATP (Cream Dilution), Pearl Dilution, Sabinol, or combinations thereof, and the edited chromosomal sequence comprises at least one mutation such that the sequence is modified and the expressed protein comprises at least one amino acid change.

7. The genetically modified equine of claim 6, wherein the equine has a different coat color or coat pattern than an equine in which the chromosomal region is not edited.

8. The genetically modified equine of claim 3, wherein the gene is HERDA, HYPP, Overo, GBE, JEB, PSSM, MSTN, RER, DNA-PKcs, cRALBP, MYO5A or combinations thereof, and the edited chromosomal sequence comprises at least one mutation such that the sequence is modified or inactivated.

9. The genetically modified equine of claim 8, wherein the gene is HERDA and the equine does not exhibit phenotypic characteristics of hereditary equine regional dermal asthenia compared to equines without the chromosomal modification.

10. The genetically modified equine of claim 3, wherein the protein is MSTN and its variants thereof, and the edited chromosomal sequence comprises at least one mutation such that the sequence is modified and the expressed protein comprises at least one amino acid change.

11. The genetically modified equine of claim 10, wherein the amino acid change results in increased athletic performance of the equine compared to equines without the amino acid change.

12. The genetically modified equine of claim 10, wherein the protein variants are MSTN-C, MSTN-T and MSTN-C/T, and the edited chromosomal sequence comprises at least one mutation such that the sequence is inactivated and the protein variants are either not produced or are not functional.

13. The genetically modified equine of claim 3, wherein the gene is HYPP and the equine does not exhibit phenotypic characteristics of Hyperkalemic Periodic Paralysis Disease.

14. The genetically modified equine of claim 3, wherein the gene is Overo and the genetic modification results in a genetic mutation of AG to TC.

15. The genetically modified equine of claim 1, wherein the equine is heterozygous or homozygous for the edited chromosomal sequence.

16. The genetically modified equine of claim 1, wherein the equine is an embryo, a foal, or an adult.

17. An equine embryo, the embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

18. The equine embryo of claim 17, wherein the chromosomal sequence comprises a gene chosen from Extension (Black/Red Factor), Agouti, MC1R, Gray Modifier, Champagne Dilution, Tobiano, Silver Dilution, MATP (Cream Dilution), Pearl Dilution, Sabinol, HERDA, HYPP, Overo, GBE, JEB, PSSM, MSTN, RER, DNA-PKcs, cRALBP, MYO5A and combinations thereof.

19. A genetically modified equine cell comprising at least one edited chromosomal sequence.

20. The genetically modified equine cell of claim 19, wherein the edited chromosomal sequence comprises a gene chosen from Extension (Black/Red Factor), Agouti, MC1R, Gray Modifier, Champagne Dilution, Tobiano, Silver Dilution, MATP (Cream Dilution), Pearl Dilution, Sabinol, HERDA, HYPP, Overo, GBE, JEB, PSSM, MSTN, RER, DNA-PKcs, cRALBP, MYO5A and combinations thereof and combinations thereof.
